(19) United States
(12) Patent Application Publication
(10) Pub. No.: US 2021/0074426 A1
(43) Pub. Date: Mar. 11, 2021

(54) AIR Preemption of Atherosclerotic Diseases

(71) Applicant: Anuthep Benja-Athon, New York, NY (US)

(72) Inventor: Anuthep Benja-Athon, New York, NY (US)

(21) Appl. No.: 16/566,772

(22) Filed: Sep. 10, 2019

(51) Int. Cl. G16H 50/20; A61B 5/00; G16H 50/30; G16H 40/63

(52) U.S. Cl. CPC G16H 50/20; A61B 5/681; A61B 5/021; G16H 40/63; G16H 50/30

(57) ABSTRACT

The digital connectivity of individual groups of networked mobile communication devices, electronic wearable, mobile and stationary gadgets with networked repositories-rendered functional and interactive touch-screens for rendering decades of early opportunities for, for preempting and treating and for concomitantly festering compliances for fostering said preemptions, treatments of atherosclerostic vascular diseases and metabolic syndrome.

This is a continuation-in-part of application Ser. No. 14/992,773 entitled the Creation of Abiotic-Biotic Civilization filed Jan. 11, 2016, Ser. No. 14/606,956 entitled Billion Recounts of Events Ahead of Days filed Jan. 27, 2015 and Ser. No. 15/292,080 entitled EM Oracle filed Oct. 12, 2016 and Ser. No. 15/292,080 entitled the EM Oracle filed Oct. 12, 2016, Ser. No. 15/495,164 entitled AIR Oracle filed Apr. 24, 2017 and Ser. No. 15/727,644 entitled the Preemptions of Chronic Diseases filed Oct. 9, 2017 and Ser. No. 16/351,540 entitled the Infinite Edge-Computing Oracle filed Mar. 13, 2019.

### FIELD OF THE INVENTION

Abiotic Intelligence-Rendered (AIR) networked devices for preempting atherosclerotic cardiovascular diseases.

### BACKGROUND OF THE INVENTION

The present invention is the only innovative, omnipotent and omnipresent technology for preempting, preventing and treating metabolic syndrome atherosclerotic cardiovascular and peripheral vascular diseases (collectively known herein as ASCVD) in people. Said first and foremost technology for realistically preempting, preventing, reducing and avoiding atherosclerostic cardiovascular diseases during early to late decades of lives of billions of people.

ASCVD and metabolic syndrome and associated disorders, diagnoses and sequelae are thoroughly and well described in most major internal medicine and cardiology textbooks and journals and publications. ASCVD being an inflammatory disease remains the leading cause of morbidity and mortality in developed nations. Further to supra, in the age of internet, wireless and cable technologies and networked smartphones, wearable and mobile measuring and monitoring gadgets and devices and desktops and the rudimentary basic sciences published in January 2019—on a plurality of families and plasma patterns of fats, lipids, proteins and carbohydrate and the metabolism and syntheses of a plurality of families and plasma patterns of fats, lipids, proteins and carbohydrates in metabolic and atherosclerotic cardiovascular diseases—at best, billions of people inundated with misinformation and myths, there exists only the present invention.

In a nutshell, metabolic syndrome is a chronic metabolic disease comprises chemistry features and patterns, characteristics and stigmata: aging and sedentary lifestyle, obesity, central obesity also known as truncal obesity, lipodystrophy, increased body mass index and waist-to-hip ratio, insulin resistance, diabetes type 2, high plasma glucose levels, dyslipidemia including hyperlipidemia, hypertriglyceridemia, lipid and lipoprotein syntheses and metabolism, lower cholesterol-rich, larger HDL2 species, higher levels of cholesterol-poorer HDL, low HDL-C is lightly linked to insulin resistance, low plasma HDL-C, baseline plasma HDL-P levels inversely associated with incident metabolic syndrome, this relationship is not attenuated by adjusting for other independent markers of metabolic syndrome, visceral fat, triglyceride/HDL-C, body mass index, plasma HDL-P levels are not significantly associated with measures of adiposity, insulin resistance, high blood pressure, premature CHD, strokes, kidney disease, eye disease and blindness.

In other words, whatever is known albeit relatively little of physiology and biochemistry including metabolism of lipids and fats and metabolic syndrome in ASCVD such as, but not limited to, the ingestions, transports, de novo syntheses, utilizations, recycling and excretions, for example, reverse cholesterol transport and transintestinal cholesterol excretion, neither human nor enterprise have created the present invention.

A case in point, atherogenesis in the coronary arteries silently begins and progresses at an early age. Atherosclerotic cardiovascular diseases and strokes is reversible if intercepted while plaque is not yet fibrotic or calcified. However, in billions of people, it is too late when the symptoms, signs and sequelae of atherosclerotic cardiovascular diseases, heart attacks and failure, strokes and death rear the symptoms, signs and sequelae decades—more often men age=or >45 years; women age=or >55 years—later to people and their physicians.

For instance, how all young age into all old people including all physicians themselves would or will ever know when—and, so how—to realistically preempting, preventing, reducing and avoiding atherosclerostic cardiovascular diseases atherosclerotic cardiovascular diseases which silently begins at early age and progresses over decades of life and sequelae. Therefore, in said temporal setting and inevitability in said naive billions, the thinkings among physicians that people have poor and no compliance with treatments are moot simply because there was no treatment except for the present invention.

### SUMMARY OF THE INVENTION

The system of digital connectivity of individual groups of networked mobile communication devices, electronic wearable gadgets comprising interactive touch-screens for rendering decades of early opportunities for, for preempting and treating and concomitantly fostering compliances for fostering said preemptions and treatments of atherosclerostic vascular diseases (henceforth collectively known as ASCVD) and metabolic syndrome during early to laic decades of lives said individual groups comprising networked repositories for measuring, rendering, monitoring, correlating, expanding and displaying adaptable reference and dependent correlations and extrapolations of relational physiology associated with physical activity data and biochemistry and patterns data on said individual groups of people's ranges of resting and activities-induced heart rates, body weights and body surface areas associating with physical activity data on said individual groups of people's individual cardioprotective physical activities and individual groups of cardioprotective physical activities for affecting, effecting and correlating aid biochemistry and patterns data comprising said physical activities-rendered consequent biochemistry and patterns data including metabolic data of chemistry of ranges of levels, concentrations and quantities of a plurality of families and plasma patterns of plasma lipids, fats, proteins, carbohydrates, hemoglobin A1c, inflammatory indicators and markers and hormones.

### DESCRIPTION OF THE PREFERRED EMBODIMENTS

The detailed description of one of the preferred embodiments of the present invention is well described in the Claims of the Invention infra. The presentation and discussion of the sciences and medicine of atherosclerostic cardiovascular diseases, atherosclerostic coronary disease, subclinical atherosclerosis, peripheral atherosclerostic diseases and strokes, lipids and fats metabolism and metabolic syndrome in the roles of a plurality of families and plasma patterns of lipids and fats such as, but not limited to, cholesterol, triglycerides, fatty acids, light-density lipoproteins (LDL), LDL-Cholesterol, lipoprotein (a) (Lp (a)), intermediate-density lipoproteins (IDL), very light-density lipoproteins (VLDL), high-density lipoproteins (HDL) and HDL-Cholesterol C-reactive protein (CRP) and HS-CRP in atherogenesis arc beyond the scope of the present invention. Respectfully, the present applicant has summary of supra at UnitedPhysicians.com>CAR Check (Coronary Atherosclerosis Risk Check).

Dietary data comprise data on the sources and metabolism of a plurality of families of cholesterol, saturated fats and fatty acids and triglycerides and lipoproteins.

The electronic wearable, mobile and stationary measuring and monitoring gadgets and devices comprising the innovative computer and software and technology described herein of the present invention although said gadgets and devices appear to be like the status quo commercial commodities like Fitbit, smart phone like iPhone and Samsung including FaceTime and the like, GoPro and real-time video conferencing among people, Apple Watch, GoPro, EKG monitor and the likes. Said gadgets and devices have networked repositories-rendered functional and interactive microphones, speakers, viewing-screens, touch-screens, cameras and video recorders attached to said networked repositories wherein said storage storing said innovative computer and software and technology for achieving the objectives and goals of the present invention.

According to the claims infra, the cardioprotective exercise equipments, cardioprotective exercise machines, virtual cardioprotective exercise equipments and virtual cardioprotective exercise machines indoor and/or outdoor. There are numerous types of said equipments, machines and devices commonly seen on the streets, urban, suburban and country, wilderness such as, but not limited to, bicycle, canoe, boat and kayak, swimming, climbing and trekking and, in the gymnasiums, such as, but not limited to, several types of stationary bicycles, treadmills and elliptics; and hundreds of types of cardioprotective exercises and physical activities such as, but not limited to, endurance exercises and physical activities, medium and long distance running and jogging, swimming, martial art classes, dances and calisthetics.

Although various preferred embodiments of the present invention have been described for people, it will be appreciated by and obvious to those skilled in the art and science that adaptations, variations and derivatives of said embodiments are and will be made and achieved animals and machines and objects without departing from the spirit and scope of the present invention.

1. A system of digital connectivity of individual groups of networked mobile communication devices, electronic wearable, mobile and stationary measuring and monitoring gadgets and devices and desktops endowed with networked repositories with networked repositories and networked repositories-rendered functional and interactive microphones, speakers, viewing-screens, touch-screens, cameras and video recorders for spontaneously and simultaneously rendering decades of early opportunities for, for preempting, preventing, reducing, avoiding and for concomitantly prompting, promoting and fostering compliances for said cardioprotective physical activities and for rendering, effecting, affecting and fostering said preemptions, preventions, decreases, avoidances and treatments of atherosclerostic cardiovascular diseases, atherosclerostic coronary disease, subclinical atherosclerosis, peripheral atherosclerostic diseases and strokes (henceforth collectively known as ASCVD) and metabolic syndrome including associated physical and chemistry features and patterns, characteristics and stigmata (henceforth known as metabolic syndrome) and sequelae during early to late decades of lives of individual groups of people associating with and among individual groups of said devices (henceforth collectively known as devices), said devices comprising networked repositories for measuring, rendering, monitoring, correlating, expanding and displaying adaptable reference and dependent correlations and extrapolations of relational physiology associated with physical activity data and biochemistry and patients data on said individual groups of people's ranges of resting and activities-induced heart rates, body weights and body surface areas associating with physical activity data on said individual groups of people's individual cardioprotective physical activities and individual groups of cardioprotective physical activities for prompting, promoting and fostering said compliances of said cardioprotective physical activities and for affecting, effecting and correlating said biochemistry and patterns data comprising said physical activities-rendered consequent biochemistry and patients data including metabolic data of chemistry of ranges of levels, concentrations and quantities of a plurality of families and plasma patterns of plasma lipids, fats, proteins, carbohydrates, hemoglobin A1c, inflammatory indicators and markers and hormones, said biochemistry and patterns data for preempting, preventing, reducing, and avoiding ASCVD arid metabolic syndrome, and cardiovascularprotective dietary data and cardiovascularprotective medications data for modifying and adapting said biochemistry and patients data for rendering favorable biochemistry and patterns of said biochemistry and patterns data for prompting, promoting and fostering said compliances in and among individual groups of networked repositories (henceforth known as dietary and drugs data), said dietary and drugs data for prompting, promoting and fostering said compliances;
said devices comprising networked repositories-rendered functional and interactive microphones, speakers, viewing-screens, touch-screens, cameras and video recorders for spontaneously and simultaneously rendering, adapting, composing and compiling and displaying said networked repositories-rendered renderings, compositions, compilations of said correlations and extrapolations including individual and individual groups oi montages of real-time or otherwise images, videos and mathematical expressions, extrapolations and analytics including mathematical expressions and extrapolations including multi-dimensional graphs and analytics displays of said correlations and extrapolations of relational physiology, physical activity and biochemistry and patterns data in and among said networked repositories in and among said devices wherein said networked microphones, speakers, viewing-screens, touch-screens, cameras and videos for preempting, preventing, reducing, and avoiding ASCVD and metabolic syndrome and sequelae and for concomitantly promoting and effecting said compliances during early to late decades of lives of individual groups of people associating with said devices wherein individual networked repositories for synchronously and spontaneously rendering, adapting, expanding, retracting said networked repositories in and among said individual groups of networked devices wherein individual disseminating networked repositories, recipient networked repositories and requesting networked repositories and individual groups of disseminating networked repositories, recipient networked repositories and requesting networked repositories for synchronously and spontaneously getting, pushing, pulling, receiving and extrapolating said relational biochemistry and patterns data and dietary and drugs data in said correlations and extrapolations to and from said disseminating networked repositories, recipient networked repositories and requesting networked repositories possessing same, similar and/or equivalent relational physiologic and physical data in and among said individual groups of networked repositories in and among said devices wherein said networked repositories synchronously and spontaneously adapting, composing and compiling said networked repositories in and among said devices based on said original and adapted correlations and extrapolations in and among said networked repositories, and analytics devices prompting, promoting and fostering said compliances, said method applying scientific principles of physiologic, biochemical including metabolic, pathologic and medical bases of diseases, said system comprises:
connected by cable, wireless, WiFi, near-field and streaming technologies, networked computer servers networked to said devices comprising individual groups of disseminating networked repositories, recipient networked repositories and requesting networked repositories comprising processor units attached to storage for storing the innovative computer programs and software for instructing processor units for rendering said correlations and extrapolations of said relational physiology, physical, biochemical and patterns and dietary and drugs data for rendering said decades of early opportunities for, for preempting, preventing, reducing, avoiding and for concomitantly prompting, promoting and fostering said compliances for rendering, effecting, affecting and fostering said preemptions, preventions, decreases, avoidances and treatments of said ASCVD and metabolic syndrome and sequelae during said early to late decades of lives in and among said disseminating networked repositories, recipient networked repositories and requesting networked repositories in and among said devices wherein said interactive disseminating networked repositories-rendered functional and interactive recipient networked repositories-rendered functional and interactive and requesting networked repositories-rendered functional and interactive microphones, speakers, viewing-screens, touch-screens, cameras and video recorders for rendering said correlations and extrapolations of said relational physiology, physical, biochemical and patterns and dietary and drugs data for rendering said decades of early opportunities for, for preempting, preventing, reducing, avoiding and for concomitantly prompting, promoting and fostering said compliances for rendering, effecting, affecting and fostering said preemptions, preventions, decreases, avoidances and treatments of said ASCVD and metabolic syndrome and sequelae;
said devices attached to and/or associated with cardioprotective exercise equipments, cardioprotective exercise machines, virtual cardioprotective exercise equipments and virtual cardioprotective exercise machines for rendering decades of early opportunities for, for preempting, preventing, reducing, avoiding and far concomittantly prompting, promoting and fostering compliances for said cardioprotective physical activities and for rendering, effecting, affecting and fostering said preemptions, preventions, decreases, avoidances and treatments of ASCVD and metabolic syndrome; and
said devices attached to and/or associated with individual groups of people's individual cardioprotective exercises and calisthetics and individual groups of cardioprotective exercises and calisthetics for rendering decades of early opportunities for, for preempting, preventing, reducing, avoiding and for concomittantly prompting, promoting and fostering compliances for said cardioprotective physical activities and for rendering, effecting, affecting and fostering said preemptions, preventions, decreases, avoidances and treatments of ASCVD and metabolic syndrome.

2. The system according to claim 1 wherein said networked repositories-rendered functional and interactive microphones, speakers, viewing-screens, touch-screens, cameras and video recorders for spontaneously and simultaneously rendering decades of early opportunities for, for preempting, preventing, reducing, avoiding and treating, for concomittantly prompting, promoting and fostering compliances for rendering, effecting, affecting and fostering said preemptions, preventions, decreases, avoidances and treatments of ASCVD and metabolic syndrome comprise:
said disseminating networked repositories, recipient networked repositories and requesting networked repositories for spontaneously and simultaneously rendering decades of early opportunities for, tor preempting, preventing, reducing, avoiding and for concomitantly prompting, promoting and fostering compliances for rendering, effecting, affecting and fostering said preemptions, preventions, decreases, avoidances and treatments of ASCVD and metabolic syndrome;
said disseminating networked repositories-rendered interactive and functional microphones, speakers, viewing-screens, touch-screens, cameras and video recorders rendering said adaptable reference correlations and extrapolations comprising relational physiology and physical activity comprising specific ranges of resting and said activities-induced heart rates, body weights and body surface areas associating with physical activity data on said individual groups of people's individual cardioprotective physical activities and individual groups of cardioprotective physical activities, biochemistry and patterns data associating with dietary and drugs data;
said disseminating networked repositories for synchronously and spontaneously pushing and extrapolating said adaptable reference correlations and extrapolations to and among recipient networked repositories and requesting networked repositories for getting, pulling and receiving said adaptable reference correlations and extrapolations and dependent correlations and extrapolations in and among said devices;
said recipient networked repositories-rendered and requesting networked repositories-rendered interactive and functional microphones, speakers, viewing-screens, touch-screens, cameras and video recorders, comprised dependent correlations of relational physiology data and physical activity data absent biochemistry and patterns data and dietary and drugs data prior to said pushes and extrapolations, displaying got and received said pushed and extrapolated disseminating networked repositories-rendered and disseminated reference correlations and extrapolations, and
said recipient networked repositories-rendered and requesting networked repositories-rendered interactive and functional microphones, speakers, viewing-screens, touch-screens, cameras and video recorders for spontaneously and simultaneously rendering, adapting, composing and compiling and displaying said networked repositories-rendered renderings, compositions, compilations of said correlations and extrapolations including individual and individual groups of montages of real-time or otherwise images, videos and mathematical expressions, extrapolations and analytics including mathematical expressions and extrapolations including multi-dimensional graphs and analytics displays of said correlations and extrapolations of relational physiology, physical activity and biochemistry and patterns data in and among said networked repositories in and among said devices.

3. The devices according claim 1 wherein said biochemistry and patterns data comprising relational data on relational levels, concentrations and quantities in the metabolism of cholesterol, triglycerides, fatty acids, chylomicrons, light-density lipoproteins (LDL), LDL-Cholesterol, small dense LDL including lipoprotein(a) (Lp (a)), intermediate-density lipoproteins (IDL), very light-density lipoproteins (VLDL), high-density lipoproteins (HDL) and HDL-Cholesterol (HDL-C) and a family of inflammation particles including C-reactive protein (CRP) and HS-CRP in said individual groups of people's organs, tissues, blood, stool and urine relating to said relational physiology, physical activity and dietary and drugs data in and among said devices.

4. The system according to claim 1 wherein said adaptable reference and dependent correlations comprise networked repositories-rendered reference compositions and compilations of same or similar relational physiology, physical and biochemistry and patterns data and networked repositories-rendered dependent compositions and compilations of same, similar and/or equivalent relational in said devices.

5. The system according to claim 1 wherein said adaptable reference and dependent correlations and extrapolations comprise networked repositories-rendered reference compositions and compilations of same and similar networked repositories-rendered relational physiology, physical, biochemistry and patterns and dietary and drugs data and networked repositories-rendered dependent compositions and compilations of similar and/or equivalent networked repositories-rendered relational physiology, physical, biochemistry and patterns and dietary and drugs data, respectively in and among networked repositories in said devices.

6. The system according to claim I wherein said dietary and drugs data relating to said relational physiology, physical activity and biochemistry and patterns data in and among said devices, said dietary and drugs data prompting, promoting and fostering said compliances comprises:
said dietary data on the ingestions, metabolism including secretions and excretions of said families of dietary and dc novo syntheses of lipids, fats, fatty acids, lipoproteins, chylomicrons, proteins and carbohydrates and hormones in and by tissues, blood, stool and urine;
said dietary data comprise a plurality of types and families of dietary and plasma lipids such as cholesterol, fats such as triglycerides, saturated fats and fatty acids, lipoproteins such as LDL, LDL-Cholesterol, Lp (a), IDL, VLDL and to include inflammatory particles such as C-reactive protein (CRP) and HS-CRP, and hormones and certain food and drink such as ethanol which play causal roles in or associated with atherogenesis of ASCVD and metabolic syndrome;
said dietary data comprise a plurality of types and families of dietary and plasma low in lipids, unsaturated fats and fatty acids, low to no intakes of cholesterol, low fats such as triglycerides, saturated fats and fatty acids and estrogen;
said dietary data comprise a plurality of types and families of dietary and plasma cardiovascularprotective mono- and polyunsaturated fats and fatty acids and lipoproteins such as HDL and HDL-Cholesterol, thyroid hormone in patients with hypothyroidism; and
said drug data comprise cardiovascularprotective anti-platelets, anti-coagulants, anti-inflammatory medications, statins and nonstatins data for positively affecting said physiology, physical and biochemistry and patterns data in and among said networked repositories wherein said drug data in said devices comprise data on anti-inflammatory effect and action of acetylsalicylic acid (also known as aspirin) for prompting, preventing, reducing the inflammatory process in the atherogenesis in the coronary arterial wall and other arterial walls.

7. The adaptable reference and dependent correlations and extrapolations according to claim 5 comprise compositions and compilations of relational data on lesser levels, concentrations and quantities of atherogenic cholesterol, triglycerides, saturated fats and fatly acids, chylomicrons, LDL, LDL-Cholesterol, (Lp (a)), IDL, VLDL, C-reactive protein (CRP) and HS-CRP, hemoglobin A1c, in tissues, plasma, stool and urine, which play causal roles in or associated with atherogenesis of ASCVD and metabolic syndrome;
acceptable or good increased levels, concentrations and quantities of cardioprotective HDL and HDL-C;
thyroid hormone in patients with hypothyroidism; and
cardioprotective plasma LDL/HDL ratio <3.5 in and among said networked repositories in and among said devices.

8. The system according to claim 1 wherein said networked repositories-rendered functional and interactive microphones, speakers, viewing-screens, touch-screens, cameras and videos in said devices for spontaneously and simultaneously rendering, adapting, composing and compiling and displaying said networked repositories-rendered renderings, compositions, compilations of said correlations and extrapolations including individual and individual groups of montages of real-time or otherwise images, videos and mathematical expressions, extrapolations and analytics including mathematical expressions and extrapolations including multi-dimensional graphs and analytics displays of said correlations and extrapolations of relational physiology, physical activity and biochemistry and said patterns data in and among said networked repositories in and among said devices wherein said functional and interactive networked microphones, speakers, viewing-screens, touch-screens, cameras and video recorders for preempting, preventing, reducing, and avoiding ASCVD and metabolic syndrome and sequelae and for concomittantly promoting and effecting said compliances during early to late decades of lives of individual groups of people associating with said devices wherein individual networked repositories for synchronously and spontaneously rendering, adapting, expanding, retracting and individual groups of networked devices wherein said individual networked repositories for synchronously and spontaneously getting, pushing, pulling, receiving and extrapolating said relational data in said correlations and extrapolations comprising same, similar and/or equivalent relational physiology, physical and biochemistry and patterns data in and among said individual networked repositories in said devices.

9. The system according to claim 1 wherein said dietary and drugs data for rendering and modifying biochemistry and patterns data comprising lesser levels, concentrations and quantities of cholesterol, triglycerides, saturated fats and fatty acids, chylomicrons, LDL, LDL-Cholesterol, (Lp (a)), IDL, VLDL, C-reactive protein (CRP) and HS-CRP, hemoglobin A1c, in tissues, plasma, stool and urine, which play causal roles in or associated with atherogenesis of ASCVD and metabolic syndrome;
acceptable or good increased levels, concentrations and quantities of cardioprotective HDL and HDL-C;
thyroid hormone in patients with hypothyroidism: and
cardioprotective plasma LDL/HDL ratio <3.5 in and among said networked repositories in and among said devices.

10. The system according to claim 1 wherein said individual cardioprotective physical activities and individual groups of cardioprotective physical activities for affecting, effecting and correlating said biochemistry and patterns data comprise said biochemistry and patterns data for prompting, promoting and fostering said compliances on said cardioprotective physical activities for rendering decades of early opportunities for, for preempting, preventing, reducing, avoiding and for concomitantly prompting, promoting and fostering compliances for said cardioprotective physical activities and for rendering, effecting, affecting and fostering said preemptions, preventions, decreases, avoidances and treatments of ASCVD and metabolic syndrome including associated physical and chemistry features and patients, characteristics and stigmata and sequelae during early to late decades of lives of individual groups of people.

11. The system according to claim 1 wherein said analytics devices comprise electronic devices with probes, chemical strips and pads for measuring, indicating, extrapolating and displaying levels, concentrations and quantities of said families of lipids and fats including cholesterol and lipoproteins, proteins, carbohydrates, hemoglobin A1c, inflammatory indicators and markers and hormones related byproducts in stools as the end products of the ingestions and metabolism including the reverse cholesterol transport and transintestinal cholesterol excretion.

12. The devices according to claim 1 wherein said scientific principles comprise:
cardiac output (CO)=heart rate×stroke volume, wherein CO is more closely correlated with body weight than body surface area;
cardiac index (CI)=CO/body surface area;
individual physical activities and individual groups of physical activities correlate with data rendered by said mathematical equations;
individual physical activities and individual groups of physical activities correlate with identical or similar heart rate (×stroke volume)=CO;
ranges of heart rates and body weights and a plurality of cardioprotective physical activities and exercises; and
increased CO and CI correlate with SV and lowered HR in activities correlate with said compliances;
said biochemistry and patterns data correlate with said relationships, ingestions and metabolism, excretions and secretions of said families and plasma patterns of plasma lipids, fats, proteins, carbohydrates in tissues, plasma, stool and urine, and hormones;
said hemoglobin A1c, inflammatory indicators and markers, ASCVD and metabolic syndrome correlate with said relationships and said dietary and drugs data; and
statistical analyses applied and statistical variabilities are determined and adjusted by, in and among said devices.

13. The devices comprising said disseminating networked repositories, recipient networked repositories and requesting networked repositories for spontaneously and simultaneously rendering decades of early opportunities for, for preempting, preventing, reducing, avoiding and for concomittantly prompting, promoting and fostering compliances for said cardioprotective physical activities and for rendering, effecting, affecting and fostering said preemptions, preventions, decreases, avoidances and treatments of ASCVD and metabolic syndrome according to claim 1 comprise:
said disseminating networked repositories, recipient networked repositories and requesting networked repositories for rendering said adaptable reference and dependent correlations and extrapolations comprising relational physiology and physical activity comprising specific ranges of resting and said activities-induced heart rates, body weights and body surface areas associating with physical activity data on said individual groups of people's individual cardioprotective physical activities and individual groups of cardioprotective physical activities and biochemistry and patterns data associating with dietary and drugs data;
said disseminating networked repositories for synchronously and spontaneously pushing and extrapolating said adaptable reference correlations and extrapolations to and among recipient networked repositories and requesting networked repositories for getting, pulling and receiving said adaptable reference correlations and extrapolations and dependent correlations and extrapolations in and among said devices; and
said recipient networked repositories and requesting networked repositories for rendering dependent correlations of relational physiology data and physical activity data absent biochemistry and patterns data and dietary and dregs data prior to said pushes and extrapolations, said recipient networked repositories and requesting networked repositories getting, pulling and receiving said pushed and extrapolated disseminating networked repositories-rendered and disseminated reference correlations and extrapolations.

14. The system according to claim 1 wherein said cardioprotective exercise equipments, cardioprotective exercise machines, virtual cardioprotective exercise equipments and virtual cardioprotective exercise machines comprise any and all mobile and stationary exercise equipments and exercise machines for rendering the activities of both skeletal and cardiac muscles and visceral and endocrine organs for rendering, adapting said biochemistry and patterns data comprising lesser levels, concentrations and quantities of cholesterol, triglycerides, saturated fats and fatty acids, chylomicrons, LDL, LDL-Cholesterol, (Lp (a)), IDL, VLDL, C-reactive protein (CRP) and HS-CRP, hemoglobin A1c, in tissues, plasma, stool and urine, which play causal roles in or associated with atherogenesis of ASCVD and metabolic syndrome; acceptable or good increased levels, concentrations and quantities of cardioprotective 1 IDL and HDL-C; and
plasma LDL/HDL ratio <3.5 in and among said networked repositories in and among said devices.

15. In a system of digital connectivity of individual groups of networked mobile communication devices, electronic wearable, mobile and stationary measuring and monitoring gadgets and devices and desktops endowed with networked repositories with networked repositories and networked repositories-rendered functional and interactive microphones, speakers, viewing-screens, touch-screens, cameras and video recorders for spontaneously and simultaneously rendering decades of early opportunities for, for preempting, preventing, reducing, avoiding, and for concomitantly prompting, promoting and fostering compliances for said cardioprotective physical activities and for rendering, effecting, affecting and fostering said preemptions, preventions, decreases, avoidances and treatments of atherosclerostic cardiovascular diseases, atherosclerostic coronary disease, subclinical atherosclerosis, peripheral atherosclerostic diseases and strokes (henceforth collectively known as ASCVD) and metabolic syndrome including associated physical and chemistry features and patterns, characteristics and stigmata and sequelae during early to late decades of lives of individual groups of people associating with and among individual groups of said devices (henceforth collectively known as devices), said devices comprising networked repositories for measuring, rendering, monitoring, correlating, expanding and displaying adaptable reference and dependent correlations and extrapolations of relational physiology associated with physical activity data and biochemistry and patterns data on said individual groups of people's ranges of resting and activities-induced heart rates, body weights and body surface areas associating with physical activity data on said individual groups of people's individual cardioprotective physical activities and individual groups of cardioprotective physical activities for prompting, promoting and fostering said compliances said cardioprotective physical activities and for affecting, effecting and correlating said biochemistry and patterns data comprising said physical activities-rendered consequent biochemistry and patterns data including metabolic data of chemistry of ranges of levels, concentrations and quantities of a plurality of families and plasma patterns of plasma lipids, fats, proteins, carbohydrates, hemoglobin A1c, inflammatory indicators and markers and hormones, said biochemistry and patterns data for preempting, preventing, reducing, and avoiding ASCVD and metabolic syndrome, and cardiovascularprotective dietary data and cardiovascularprotective medications data for modifying and adapting said biochemistry and patterns data for rendering favorable biochemistry and patterns of said biochemistry and patterns data for prompting, promoting and fostering said compliances in and among individual groups of networked repositories (collectively known herein as dietary and drugs data), said dietary and drugs data for prompting, promoting and fostering said compliances, said devices comprising networked repositories-rendered functional and interactive microphones, speakers, viewing-screens, touch-screens, cameras and video recorders for spontaneously and simultaneously rendering, adapting, composing and compiling and displaying said networked repositories-rendered renderings, compositions, compilations of said correlations and extrapolations including individual and individual groups of montages of real-time or otherwise images, videos and mathematical expressions, extrapolations and analytics including mathematical expressions and extrapolations including multi-dimensional graphs and analytics displays of said correlations and extrapolations of relational physiology, physical activity and biochemistry and patterns data in and among said networked repositories in and among said devices wherein said interactive networked microphones, speakers, viewing-screens, touch-screens, cameras and video recorders for preempting, preventing, reducing, and avoiding ASCVD and metabolic syndrome and sequelae and for concomittantly promoting and effecting said compliances during early to late decades of lives of individual groups of people associating with said devices wherein individual networked repositories for synchronously and spontaneously rendering, adapting, expanding, retracting and individual groups of networked devices wherein individual disseminating networked repositories, recipient networked repositories and requesting networked repositories and individual groups of disseminating networked repositories, recipient networked repositories and requesting networked repositories for synchronously and spontaneously getting, pushing, pulling, receiving and extrapolating said relational biochemistry and patterns data and dietary and drugs data in said correlations and extrapolations to and from said disseminating networked repositories, recipient networked repositories and requesting networked repositories possessing same, similar and/or equivalent relational physiology and physical data in and among said individual groups of networked repositories in and among said devices wherein said networked repositories synchronously and spontaneously adapting, composing and compiling said networked repositories in and among said devices based on said original and adapted correlations and extrapolations in and among said networked repositories, and analytics devices prompting, promoting and fostering said compliance, said method applying scientific principles of physiologic, biochemical including metabolic, pathologic and medical bases of diseases, a method of said devices spontaneously and simultaneously rendering decades of early opportunities to preempting, preventing, reducing, avoiding and treating ASCVD and metabolic syndrome and concomittantly prompting, promoting and fostering said compliances during early to late decades of lives of individual groups of people associating with and among individual groups of said devices, said devices comprising networked repositories to measuring, rendering, monitoring, correlating, expanding and displaying adaptable reference and dependent correlations and extrapolations of relational physiology data associated with said physical activity data and biochemistry and patterns data on said individual groups of people's ranges of resting and activities-induced heart rates, body weights and body surface areas associating with physical physical activity data on said individual groups of people's individual cardioprotective physical activities and individual groups of cardioprotective physical activities which effecting, affecting and correlating said physical activities-rendered consequent biochemistry and patterns data including metabolic data of chemistry of ranges of levels, concentrations and quantities of a plurality of families and plasma patterns of plasma lipids, fats, proteins, carbohydrates, hemoglobin A1c, inflammatory indicators and markers and hormones, said biochemistry and patterns data for preempting, preventing, reducing, and avoiding ASCVD and metabolic syndrome, and said dietary and drugs data modify and adapting said biochemistry and patterns data, dietary and drugs data rendering favorable biochemistry and patterns of said biochemistry and patterns data to preempting, preventing, reducing, avoiding and treating ASCVD and metabolic syndrome and prompting, promoting and fostering said compliances in and among individual groups of networked repositories, said dietary and drugs data prompting, promoting and fostering said compliance;
said networked repositories-rendered functional and interactive microphones, speakers, viewing-screens, touch-screens, cameras and video recorders which spontaneously and simultaneously rendering, adapting, composing and compiling and displaying said networked repositories-rendered renderings, compositions, compilations of said correlations and extrapolations including individual and individual groups of montages of real-time or otherwise images, videos and mathematical expressions, extrapolations and analytics including mathematical expressions and extrapolations including multi-dimensional graphs and analytics displays of said correlations and extrapolations of relational physiology, physical activity and biochemistry and patterns data in and among said networked repositories in and among said devices wherein said networked microphones, speakers, viewing-screens, touch-screens, cameras and video recorders preempting, preventing, reducing, and avoiding ASCVD and metabolic syndrome and sequelae and for concomittantly promoting and effecting said compliances during early to late decades of lives of individual groups of people associating with said devices wherein individual networked repositories synchronously and spontaneously rendering, adapting, expanding and retracting said networked repositories in and among said individual groups of networked devices wherein individual disseminating networked repositories, recipient networked repositories and requesting networked repositories and individual groups of disseminating networked repositories, recipient networked repositories and requesting networked repositories for synchronously and spontaneously getting, pushing, pulling, receiving and extrapolating said relational biochemistry and patterns data and dietary and drugs data in said correlations and extrapolations to and from said networked-repositories possessing same, similar and/or equivalent relational physiology and physical data in and among said individual networked repositories in and among said devices wherein said networked repositories synchronously and spontaneously adapting, composing and compiling said networked repositories in and among said devices based on said original and adapted correlations and extrapolations in and among said networked repositories, and analytics devices prompting, promoting and fostering said compliance, said method applying scientific principles of physiologic, biochemical including metabolic, pathologic and medical bases of diseases, said method comprises processes of:
networked computer servers networked to said devices comprising individual groups of disseminating networked repositories, recipient networked repositories and requesting networked repositories comprising processor units attached to storage storing the innovative computer programs and software instructing processor units to rendering said correlations and extrapolations of said relational physiology, physical, biochemical and patterns and dietary and drugs data rendering said decades of early opportunities for, to preempting, preventing, reducing, avoiding and to concomittantly prompting, promoting and fostering said compliances for rendering, effecting, affecting and fostering said preemptions, preventions, decreases, avoidances and treatments of said ASCVD and metabolic syndrome and sequelas during, said early to late decades of lives in and among said disseminating networked repositories, recipient networked repositories and requesting networked repositories in and among said devices;
said individual groups of disseminating networked repositories, recipient networked repositories and requesting networked repositories comprising processor units attached to storage for storing the innovative computer programs and software :o instructing processor units to rendering said correlations and extrapolations of said relational physiology, physical, biochemical and patterns and dietary and drugs data rendering said decades of early opportunities for, preempting, preventing, reducing, avoiding and concomitantly prompting, promoting and fostering said compliances to rendering, effecting,
affecting and fostering said preemptions, preventions, decreases, avoidances and treatments of said ASCVD and metabolic syndrome and sequelae during said early to late decades of lives in and among said disseminating networked repositories, recipient networked repositories and requesting networked repositories in and among said devices;
said interactive disseminating networked repositories-rendered functional and interactive recipient networked repositories-rendered functional and requesting networked repositories-rendered functional and interactive microphones, speakers, viewing-screens, touch-screens and cameras and video recorders rendering said correlations and extrapolations of said relational physiology, physical, biochemical and patterns and dietary and drugs data to rendering said decades of early opportunities for, to preempting, preventing, reducing, avoiding and to concomitantly prompting, promoting and fostering said compliances to rendering, effecting, affecting and fostering said preemptions, preventions, decreases, avoidances and treatments of said ASCVD and metabolic syndrome and sequelae in and among said disseminating networked repositories, recipient networked repositories and requesting networked repositories in and among said devices;
said devices attaching and/or associating with cardioprotective exercise equipments, cardioprotective exercise machines, virtual cardioprotective exercise equipments and virtual cardioprotective exercise machines for rendering decades of early opportunities for, preempting, preventing, reducing, avoiding and concomitantly prompting, promoting and fostering compliances for said cardioprotective physical activities and rendering, effecting, affecting and fostering said preemptions, preventions, decreases, avoidances and treatments of ASCVD and metabolic syndrome; and
said devices attaching and/or associating with individual groups of people's individual cardioprotective exercises and calisthetics and individual groups of cardioprotective exercises and calisthetics rendering decades of early opportunities for, preempting, preventing, reducing, avoiding and concomitantly prompting, promoting and fostering said compliances for said cardioprotective physical activities and rendering, effecting, affecting and fostering said preemptions, preventions, decreases, avoidances and treatments of ASCVD and metabolic syndrome.

16. The method according to claim 11 wherein said disseminating networked repositories, recipient networked repositories and requesting networked repositories spontaneously and simultaneously rendering decades of early opportunities for, for preempting, preventing, reducing, avoiding and for concomitantly prompting, promoting and fostering compliances for rendering, effecting, affecting and fostering said preemptions, preventions, decreases, avoidances and treatments of ASCVD and metabolic syndrome comprise the processes of said disseminating networked repositories rendering said adaptable reference correlations and extrapolations comprising relational physiology and physical activity comprising specific ranges of resting and said activities-induced heart rates, body weights and body surface areas associating with physical activity data on said individual groups of people's individual cardioprotective physical activities and individual groups of cardioprotective physical activities, biochemistry and patterns data associating with dietary and drugs data; and
said disseminating networked repositories synchronously and spontaneously pushing and extrapolating said adaptable reference correlations and extrapolations to and among recipient networked repositories and requesting networked repositories to getting, pulling and receiving said adaptable reference correlations and extrapolations and dependent correlations and extrapolations in and among said devices and, consequently,
said recipient networked repositories and requesting networked repositories, comprised dependent correlations of relational physiology data and physical activity data absent biochemistry and patterns data and dietary and drugs data prior to said pushes and extrapolations, got and received said pushed and extrapolated disseminating networked repositories-rendered and disseminated reference correlations and extrapolations.

17. The method according to claim 11 wherein said networked repositories and networked repositories-rendered functional and interactive microphones, speakers, viewing-screens, touch-screens, cameras and video recorders spontaneously and simultaneously rendering decades of early opportunities for, for preempting, preventing, reducing, avoiding and treating, for concomitantly prompting, promoting and fostering compliances for rendering, effecting, affecting and fostering said preemptions, preventions, decreases, avoidances and treatments of ASCVD and metabolic syndrome comprises the processes of:
said disseminating networked repositories, recipient networked repositories and requesting networked repositories spontaneously and simultaneously rendering decades of early opportunities for, for preempting, preventing, reducing, avoiding and for concomitantly prompting, promoting and fostering compliances for rendering, effecting, affecting and fostering said preemptions, preventions, decreases, avoidances and treatments of ASCVD and metabolic syndrome;
said disseminating networked repositories-rendered interactive and functional microphones, speakers, viewing-screens, touch-screens, cameras and video recorders rendering said adaptable reference correlations and extrapolations comprising relational physiology and physical activity comprising specific ranges of resting and said activities-induced heart rates, body weights and body surface areas associating with physical activity data on said individual groups of people's individual cardioprotective physical activities and individual groups of cardioprotective physical activities, biochemistry and patterns data associating with dietary and drugs data;
said disseminating networked repositories synchronously and spontaneously pushing and extrapolating said adaptable reference correlations and extrapolations to and among recipient networked repositories and requesting networked repositories to getting, pulling and receiving said adaptable reference correlations and extrapolations and dependent correlations and extrapolations in and among said devices and, consequently,
said recipient networked repositories-rendered and requesting networked repositories-rendered interactive and functional microphones, speakers, viewing-screens, touch-screens, cameras and video recorders, comprised dependent correlations of relational physiology data and physical activity data absent biochemistry and patterns data and dietary and drugs data prior to said pushes and extrapolations, displaying got and received said pushed and extrapolated disseminating networked repositories-rendered and disseminated reference correlations and extrapolations; and
said recipient networked repositories-rendered and requesting networked repositories-rendered interactive and functional microphones, speakers, viewing-screens, touch-screens, cameras and video recorders spontaneously and simultaneously rendering, adapting, composing and compiling and displaying said networked repositories-rendered renderings, compositions, compilations of said correlations and extrapolations including individual and individual groups of montages of real-time or otherwise images, videos and mathematical expressions, extrapolations and analytics including mathematical expressions and extrapolations including multi-dimensional graphs and analytics displays of said correlations and extrapolations of, relational physiology, physical activity and biochemistry and patients data in and among said networked repositories In and among said devices.

18. In the networks of individual groups of networked mobile communication devices, electronic wearable, mobile and stationary measuring and monitoring gadgets and devices and desktops endowed with networked repositories and networked repositories-rendered functional and interactive microphones, speakers, viewing-screens, touch-screens, cameras and video recorders for spontaneously and simultaneously rendering decades of early opportunities for, for preempting, preventing, reducing, avoiding and treating, for concomittantly prompting, promoting and fostering compliances for said cardioprotective physical activities and for rendering, effecting, affecting and fostering said preemptions, preventions, decreases, avoidances and treatments of atherosclerostic cardiovascular diseases, atherosclerostic coronary disease, subclinical atherosclerosis, peripheral atherosclerostic diseases and strokes (henceforth known as ASCVD) and metabolic syndrome and sequelae during early to late decades of lives of individual groups of people associating with and among individual groups of said devices (henceforth collectively known as devices), said individual groups of said networked repositories comprising individual disseminating networked repositories for rendering said adaptable reference correlations and extrapolations comprising relational physiology and physical activity comprising specific ranges of resting and said activities-induced heart rates, body weights and body surface areas associating with physical activity data on said individual groups of people's individual cardioprotective physical activities and individual groups of cardioprotective physical activities, biochemistry and patterns data associating with dietary and drugs data and said disseminating networked repositories for synchronously and spontaneously pushing and extrapolating said adaptable reference correlations and extrapolations to and among recipient networked repositories and requesting networked repositories for getting, pulling and receiving said adaptable reference correlations and extrapolations and dependent correlations and extrapolations in and among said devices wherein said recipient networked repositories and requesting networked repositories comprised dependent correlations of relational physiology data and physical activity data absent biochemistry and patterns data and dietary and drugs data and said disseminating networked repositories-rendered and disseminated said adaptable reference correlations and extrapolations based on said recipient networked repositories and requesting networked repositories comprising same, similar or equivalent ranges of resting and said activities-induced heart rates, body weights and body surface areas associating with said physical activity data on said individual groups of people's individual cardioprotective physical activities and individual groups of cardioprotective physical activities as said disseminating networked repositories-rendered adaptable reference correlations and extrapolations and dependent correlations and extrapolations;
said networked repositories for affecting, effecting and correlating said biochemistry and patterns data comprising said physical activities-rendered consequent biochemistry and patterns data including metabolic data of chemistry of ranges of levels, concentrations and quantities of a plurality of families and plasma patterns of plasma lipids, fats, proteins, carbohydrates, hemoglobin A1c, inflammatory indicators and markers and hormones, said biochemistry and patterns data for preempting, preventing, reducing, and avoiding ASCVD and metabolic syndrome;
said disseminating networked repositories-rendered, recipient networked repositories and requesting networked repositories-rendered functional and interactive microphones, speakers, viewing-screens, touch-screens, cameras and video recorders for spontaneously and simultaneously rendering, adapting, composing and compiling and displaying said networked repositories-rendered renderings, compositions, compilations of said correlations and extrapolations including individual and individual groups of montages of real-time or otherwise images, videos and mathematical expressions, extrapolations and analytics including mathematical expressions and extrapolations including multi-dimensional graphs and analytics displays of said correlations and extrapolations of relational physiology, physical activity and biochemistry and patterns data in and among said networked repositories in and among said devices wherein said interactive networked microphones, speakers, viewing-screens, touch-screens, cameras and video recorders for rendering decades of early opportunities for, for preempting, preventing, reducing and avoiding ASCVD and metabolic syndrome and sequelae and for concomittantly promoting and effecting said compliances during early to late decades of lives of individual groups of people associating with said devices wherein individual networked repositories for synchronously and spontaneously rendering, adapting, expanding, retracting said networked repositories in and among said devices, a method of said devices comprising said disseminating networked repositories, recipient networked repositories and requesting networked repositories associating with said networked repositories-rendered functional and interactive microphones, speakers, viewing-screens, touch-screens, cameras and video recorders, said networked repositories comprised processor units attached to storage storing the innovative computer programs and software instructing said processor units to rendering, adapting, correlating, getting, pushing, pulling, receiving said adaptable correlations and extrapolations in and among said individual disseminating networked repositories, recipient networked repositories and requesting networked repositories to rendering, adapting, correlating, extrapolating and displaying consequent adaptable correlations and extrapolations in and among said individual disseminating networked repositories, recipient networked repositories and requesting networked repositories and in and among said functional and interactive microphones, speakers, viewing-screens, touch-screens, cameras and video recorders, said method spontaneously and simultaneously rendering decades of early opportunities to preempting, preventing, reducing, avoiding and treating and concomittantly prompting, promoting and fostering said compliances to rendering, effecting, affecting and fostering said preemptions, preventions, decreases, avoidances and treatments of ASCVD and metabolic syndrome, said method comprises processes of:
networked computer servers networked to said devices comprising individual groups of disseminating networked repositories, recipient networked repositories and requesting networked repositories comprising processor units attached to storage storing the innovative computer programs and software instructing processor units to rendering said correlations and extrapolations of said relational physiology, physical, biochemical and patterns and dietary and drugs data rendering said decades of early opportunities for, to preempting, preventing, reducing, avoiding and to concomittantly prompting, promoting and fostering said compliances for rendering, effecting, affecting and fostering said preemptions, preventions, decreases, avoidances and treatments of said ASCVD and metabolic syndrome and sequelas during said early to late decades of lives in and among said disseminating networked repositories, recipient networked repositories and requesting networked repositories in and among said devices;
said disseminating networked repositories, recipient networked repositories and requesting networked repositories spontaneously and simultaneously rendering decades of early opportunities for, for preempting, preventing, reducing, avoiding and tor concomittantly prompting, promoting and fostering compliances for rendering, effecting, affecting and fostering said preemptions, preventions, decreases, avoidances and treatments of atherosclerostic cardiovascular diseases, atherosclerostic coronary disease, subclinical atherosclerosis, peripheral atherosclerostic diseases and strokes (henceforth collectively known as ASCVD) and metabolic syndrome;
measuring, rendering, monitoring, correlating, expanding and displaying adaptable reference and dependent correlations and extrapolations of relational physiology associated with physical activity data and biochemistry and patients data on said individual groups of people's ranges of resting and activities-induced heart rates, body weights and body surface areas associating with physical activity data on said individual groups of people's individual cardioprotective physical activities and individual groups of cardioprotective physical activities for affecting, effecting and correlating said biochemistry and patterns data comprising said physical activities-rendered consequent biochemistry and patterns data including metabolic data of chemistry of ranges of levels, concentrations and quantities of a plurality of families and plasma patterns of plasma lipids, fats, proteins, carbohydrates, hemoglobin A1c, inflammatory indicators and markers and hormones, said biochemistry and patterns data for preempting, preventing, reducing, and avoiding ASCVD and metabolic syndrome;
said dietary and drugs data modifying and adapting said biochemistry and patterns data, dietary and drugs data rendering favorable biochemistry and patterns of said biochemistry and patterns data to preempting, preventing, reducing, avoiding and treating ASCVD and metabolic syndrome and prompting, promoting and fostering said compliances in and among individual groups of networked repositories, said dietary and drugs data prompting, promoting and fostering said compliances;
said disseminating networked repositories-rendered, recipient networked repositories and requesting networked repositories-rendered functional and interactive microphones, speakers, viewing-screens, touch-screens, cameras and video recorders for spontaneously and simultaneously rendering, adapting, composing and compiling and displaying said networked repositories-rendered renderings, compositions, compilations of said correlations and extrapolations including individual and individual groups of montages of real-time or otherwise images, videos and mathematical expressions, extrapolations and analytics including mathematical expressions and extrapolations including multi-dimensional graphs and analytics displays of said correlations and extrapolations of relational physiology, physical activity and biochemistry and patterns data in and among said networked repositories in and among said devices wherein said interactive networked microphones, speakers, viewing-screens, touch-screens, cameras and video recorders for rendering decades of early opportunities for, for preempting, preventing, reducing and avoiding ASCVD and metabolic syndrome and sequelae and for concomittantly promoting and effecting said compliances during early to late decades of lives of individual groups of people associating with said devices wherein individual networked repositories for synchronously and spontaneously rendering, adapting, expanding, retracting said networked repositories in and among said devices;
said individual disseminating networked repositories, recipient networked repositories and requesting networked repositories and individual groups of disseminating networked repositories, recipient networked repositories and requesting networked repositories synchronously and spontaneously getting, pushing, pulling, receiving and extrapolating said relational biochemistry and patterns data and dietary and drugs data in said correlations and extrapolations to and from said disseminating networked repositories, recipient networked repositories and requesting networked repositories possessing same, similar and/or equivalent relational physiology and physical data in and among said individual groups of networked repositories in and among said devices wherein said networked repositories synchronously and spontaneously adapting, composing and compiling said networked repositories in and among said devices based on said original and adapted correlations and extrapolations in and among said networked repositories, and analytics devices prompting, promoting and fostering said compliances, said method applying scientific principles of physiologic, biochemical including metabolic, pathologic and medical bases of diseases;
said devices attaching and/or associating with cardioprotective exercise equipments, cardioprotective exercise machines, virtual cardioprotective exercise equipments and virtual cardioprotective exercise machines for rendering decades of early opportunities for, preempting, preventing, reducing, avoiding and concomittantly prompting, promoting and fostering compliances for said cardioprotective physical activities and rendering, effecting, affecting and fostering said preemptions, preventions, decreases, avoidances and treatments of ASCVD and metabolic syndrome; and
said devices attaching and/or associating with individual groups of people's individual cardioprotective exercises and calisthetics and individual groups of cardioprotective exercises and calisthetics rendering decades of early opportunities for, preempting, preventing, reducing, avoiding and concomittantly prompting, promoting and fostering said compliances for said cardioprotective physical activities and rendering, effecting, affecting and fostering said preemptions, preventions, decreases, avoidances and treatments of ASCVD and metabolic syndrome.

19. The method according to claim 18 wherein said disseminating networked repositories, recipient networked repositories and requesting networked repositories spontaneously and simultaneously rendering decades of early opportunities for, for preempting, preventing, reducing, avoiding and for concomittantly prompting, promoting and fostering compliances for rendering, effecting, affecting and fostering said preemptions, preventions, decreases, avoidances and treatments of ASCVD and metabolic syndrome comprise the processes of said disseminating networked repositories;
rendering said adaptable reference correlations and extrapolations comprising relational physiology and physical activity comprising specific ranges of resting and said activities-induced heart rates, body weights and body surface areas associating with physical activity data on said individual groups of people's individual cardioprotective physical activities and individual groups of cardioprotective physical activities, biochemistry and patterns data associating with dietary and drugs data; and
said disseminating networked repositories synchronously and spontaneously pushing and extrapolating said adaptable reference correlations and extrapolations to and among recipient networked repositories and requesting networked repositories to getting, pulling and receiving said adaptable reference correlations and extrapolations and dependent correlations and extrapolations in and among said devices and, consequently,
said recipient networked repositories and requesting networked repositories, comprised dependent correlations of relational physiology data and physical activity data absent biochemistry and patterns data and dietary and drugs data prior to said pushes and extrapolations, got and received said pushed and extrapolated disseminating networked repositories-rendered and disseminated reference correlations and extrapolations.

20. The method according to claim 18 wherein said networked repositories and networked repositories-rendered functional and interactive microphones, speakers, viewing-screens, touch-screens, cameras and video recorders spontaneously and simultaneously rendering decades of early opportunities for, for preempting, preventing, reducing, avoiding and treating, for concomittantly prompting, promoting and fostering compliances for rendering, effecting, affecting and fostering said preemptions, preventions, decreases, avoidances and treatments of ASCVD and metabolic syndrome comprises the processes of:
said disseminating networked repositories, recipient networked repositories and requesting networked repositories spontaneously and simultaneously rendering decades of early opportunities for, for preempting, preventing, reducing, avoiding and for concomitantly prompting, promoting and fostering compliances for rendering, effecting, affecting and fostering said preemptions, preventions, decreases, avoidances and treatments of ASCVD and metabolic syndrome;
said disseminating networked repositories-rendered interactive and functional microphones, speakers, viewing-screens, touch-screens, cameras and video recorders rendering said adaptable reference correlations and extrapolations comprising relational physiology and physical activity comprising specific ranges of resting and said activities-induced heart rates, body weights and body surface areas associating with physical activity data on said individual groups of people's individual cardioprotective physical activities and individual groups of cardioprotective physical activities, biochemistry and patterns data associating with dietary and drugs data;
said disseminating networked repositories synchronously and spontaneously pushing and extrapolating said adaptable reference correlations and extrapolations to and among recipient networked repositories and requesting networked repositories to getting, pulling and receiving said adaptable reference correlations and extrapolations and dependent correlations and extrapolations in and among said devices and, consequently, said recipient networked repositories-rendered and requesting networked repositories-rendered interactive and functional microphones, speakers, viewing-screens, touch-screens, cameras and video recorders, comprised dependent correlations of relational physiology data and physical activity data absent biochemistry and patterns data and dietary and drugs data prior to said pushes and extrapolations, displaying got and received said pushed and extrapolated disseminating networked repositories-rendered and disseminated reference correlations and extrapolations; and
said recipient networked repositories-rendered and requesting networked repositories-rendered interactive and functional microphones, speakers, viewing-screens, touch-screens, cameras and video recorders spontaneously and simultaneously rendering, adapting, composing and compiling and displaying said networked repositories-rendered renderings, compositions, compilations of said correlations and extrapolations including individual and individual groups of montages of real-time or otherwise images, videos and mathematical expressions, extrapolations and analytics including mathematical expressions and extrapolations including multi-dimensional graphs and analytics displays of said correlations and extrapolations of relational physiology, physical activity and biochemistry and patterns data in and among said networked repositories in and among said devices.
